SEQUENCE LISTING (54 sequences)

SEQ ID NO: 1 - protein, 7 aa, Human
DHNSNIR

SEQ ID NO: 2 - protein, 15 aa, Human
CVNPPTVQNAYIVSR

SEQ ID NO: 3 - protein, 18 aa, Human
DVYKAGEQVTYTCATYYK

SEQ ID NO: 4 - protein, 6 aa, Human
GEFVWK

SEQ ID NO: 5 - protein, 6 aa, Human
ELISER

SEQ ID NO: 6 - protein, 7 aa, Human
LHIMAGR

SEQ ID NO: 7 - protein, 13 aa, Human
LIAEEGVDSLNVK

SEQ ID NO: 8 - protein, 13 aa, Human
GSATKDFSVFFQK

SEQ ID NO: 9 - protein, 11 aa, Human
SEVAKDFEPER

SEQ ID NO: 10 - protein, 9 aa, Human
VQSYEFLQK

SEQ ID NO: 11 - protein, 9 aa, Human
AFVEATFQR

SEQ ID NO: 12 - protein, 12 aa, Human
MDVGGLSDPYVK

SEQ ID NO: 13 - protein, 9 aa, Human
LTVCILEAK

SEQ ID NO: 14 - protein, 20 aa, Human
HTFMGVVSLGSPSGEVSHPR

SEQ ID NO: 15 - protein, 10 aa, Human
LSYTCEGGFR

SEQ ID NO: 16 - protein, 20 aa, Human
LVQYRGEVQAMLGQSTEELR

SEQ ID NO: 17 - protein, 19 aa, Human
YSLFCLGFGFDVSYAFLEK

SEQ ID NO: 18 - protein, 15 aa, Human
EEMTKNQVSLTCLVK

SEQ ID NO: 19 - protein, 17 aa, Human
HPVHRDFLGFYVVDSHR

SEQ ID NO: 20 - protein, 10 aa, Human
WEAEPVYVQR

SEQ ID NO: 21 - protein, 18 aa, Human
ASEAEDASLLSFMQGYMK

SEQ ID NO: 22 - protein, 18 aa, Human
VASGSDLHLTDIDSDSNR

SEQ ID NO: 23 - protein, 13 aa, Human
TLRVQGNDHSATR

SEQ ID NO: 24 - protein, 14 aa, Human
YLIDHPVSLTGAGR

SEQ ID NO: 25 - protein, 26 aa, Human
VTASDPLDTLGSEGALSPGGVASLLR

SEQ ID NO: 26 - protein, 17 aa, Human
AFLLTPRNQEACELSNN

SEQ ID NO: 27 - protein, 12 aa, Human
SVSEINPTTQMK

SEQ ID NO: 28 - protein, 7 aa, Human
NPLVWVH

SEQ ID NO: 29 - protein, 16 aa, Human
ANTVQEATFQMELPKK

SEQ ID NO: 30 - protein, 13 aa, Human
NGIDIYSLTVDSR

SEQ ID NO: 31 - protein, 10 aa, Human
LALDNGGLAR

SEQ ID NO: 32 - protein, 21 aa, Human
AEATTLHVAPQGTAMAVSTFR

SEQ ID NO: 33 - protein, 20 aa, Human
LDGICWQVRQLYGDTGVLGR

SEQ ID NO: 34 - protein, 10 aa, Human
KLDGICWQVR

SEQ ID NO: 35 - protein, 12 aa, Human
GITAMAWGVEEK

SEQ ID NO: 36 - protein, 9 aa, Human
YELTGKFER

SEQ ID NO: 37 - protein, 20 aa, Human
EDAQVAAEILEIADTPSGDK

SEQ ID NO: 38 - protein, 24 aa, Human
ASFTCTCKPGWQGEKCEFDINECK

SEQ ID NO: 39 - protein, 24 aa, Human
SCVNAIPDQCSPLPCNEDGYMSCK

SEQ ID NO: 40 - protein, 8 aa, Human
FSAEFDFR

SEQ ID NO: 41 - protein, 19 aa, Human
EEAREVFENDPETDYFYPK

SEQ ID NO: 42 - protein, 14 aa, Human
HNDIRAHSCPSVWK

SEQ ID NO: 43 - protein, 13 aa, Human
KVESELIKPINPR

SEQ ID NO: 44 - protein, 22 aa, Human
LAQDQKSCEVVSVCLPLNLDTK

SEQ ID NO: 45 - protein, 20 aa, Human
GFIDGHYKDYFVPQLYSFLK

SEQ ID NO: 46 - protein, 26 aa, Human
TDLSFGSQIEFSCSEGFFLIGSTTSR

SEQ ID NO: 47 - protein, 21 aa, Human
GLEWVSAIGTAGDQYYADSVK

SEQ ID NO: 48 - protein, 20 aa, Human
LQNNENNISCVERGWSTPPK

SEQ ID NO: 49 - protein, 13 aa, Human
DIAPTLTLYVGKK

SEQ ID NO: 50 - protein, 12 aa, Human
ISMLEKIYIHPR

SEQ ID NO: 51 - protein, 11 aa, Human
SYTSHTNEIHK

SEQ ID NO: 52 - protein, 11 aa, Human
ATVVYQGERVK

SEQ ID NO: 53 - protein, 18 aa, Human
IPCSQPPQIEHGTINSSR

SEQ ID NO: 54 - protein, 25 aa, Human
HPNVFGFCRADLFYDVEALDLESPK

(19) United States
(12) Patent Application Publication
(10) Pub. No.: US 2013/0178385 A1
(43) Pub. Date: Jul. 11, 2013

(54) BIOMARKERS

(71) Applicants: Sabine Bahn, Cambridge (GB); Emanuel Schwarz, Cambridge (GB); Yishai Levin, Cambridge (GB)

(72) Inventors: Sabine Bahn, Cambridge (GB); Emanuel Schwarz, Cambridge (GB); Yishai Levin, Cambridge (GB)

(73) Assignee: CAMBRIDGE ENTERPRISE LIMITED, Cambridge (GB)

(21) Appl. No.: 13/698,833

(22) PCT Filed: May 18, 2011
(86) PCT No.: PCT/GB2011/050943

§ 371 Date: Mar. 13, 2013

(30) Foreign Application Priority Data
May 19, 2010 (GB) 1008340.0

(51) Int. Cl. G01N 33/68

(52) U.S. Cl. CPC G01N 33/6896
USPC 506/9; 436/501; 435/7.1; 435/7.92; 435/287.2; 506/39; 422/430

(57) ABSTRACT

The invention relates to a method of diagnosing or monitoring schizophrenia or other psychotic disorder.

### FIELD OF THE INVENTION

The invention relates to a method of diagnosing or monitoring schizophrenia or other psychotic disorder.

### BACKGROUND OF THE INVENTION

Schizophrenia is a psychiatric diagnosis that describes a mental disorder characterized by abnormalities in the perception or expression of reality. It most commonly manifests as auditory hallucinations, paranoid or bizarre delusions, or disorganized speech and thinking with significant social or occupational dysfunction. Onset of symptoms typically occurs in young adulthood, with approximately 0.4-0.6% of the population affected. Diagnosis is based on the patient's self-reported experiences and observed behavior. No laboratory test for schizophrenia currently exists.

Studies suggest that genetics, early environment, neurobiology, psychological and social processes are important contributory factors; some recreational and prescription drugs appear to cause or worsen symptoms. Current psychiatric research is focused on the role of neurobiology, but no single organic cause has been found. Due to the many possible combinations of symptoms, there is debate about whether the diagnosis represents a single disorder or a number of discrete syndromes.

The disorder is thought to mainly affect cognition, but it also usually contributes to chronic problems with behavior and emotion. People with schizophrenia are likely to have additional (comorbid) conditions, including major depression and anxiety disorders; the lifetime occurrence of substance abuse is around 40%. Social problems, such as long-term unemployment, poverty and homelessness, are common. Furthermore, the average life expectancy of people with the disorder is 10 to 12 years less than those without, due to increased physical health problems and a higher suicide rate.

An important utility of biomarkers for psychotic disorders is their response to medication. Administration of antipsychotics remains a subjective process, relying solely on the experience of clinicians. Furthermore, the development of antipsychotic drugs has been based on chance findings often with little relation to the background driving the observations.

Schizophrenia is treated primarily with antipsychotic medications which are also referred to as neuroleptic drugs or neuroleptics. Newer antipsychotic agents such as clozapine, olanzapine, quetiapine or risperidone are thought to be more effective in improving negative symptoms of psychotic disorders than older medication like Chlorpromazine. Furthermore, they induce less extrapyramidal side effects (EPS) which are movement disorders resulting from antipsychotic treatment.

The history of neuroleptics dates back to the late 19th century. The flourishing dye industry catalyzed development of new chemicals that lay the background to modern day atypical antipsychotics. Developments in anti-malaria, anti-histamine and anaesthetic compounds also produced various neuroleptics. The common phenomenon to all these processes is a fundamental lack of understanding of the biological mechanisms and pathways that these drugs affect, apart from the observation that they prominently block D2 receptors in the striatum.

There is therefore a pressing need for objective molecular readouts that can diagnose schizophrenia or other psychotic disorders and furthermore indicate whether a patient is responding to medication, as well as for predicting prognosis.

### SUMMARY OF THE INVENTION

According to a first aspect of the invention, there is provided the use of Complement factor H or a functional fragment or phosphorylated fragment thereof, as a biomarker for schizophrenia or other psychotic disorder, or predisposition thereto.

According to a second aspect of the invention, there is provided the use of one or more peptides selected from SEQ ID NOs 1 to 54, or a phosphorylated derivative thereof, as a biomarker for schizophrenia or other psychotic disorder, or predisposition thereto.

According to a third aspect of the invention, there is provided a method of diagnosing or monitoring schizophrenia or other psychotic disorder, or predisposition thereto, comprising detecting and/or quantifying, in a sample from a test subject, the analyte biomarkers defined herein.

According to a fourth aspect of the invention, there is provided a method of diagnosing schizophrenia or other psychotic disorder, or predisposition in an individual thereto, comprising:
(a) obtaining a biological sample from an individual;
(b) quantifying the amounts of the analyte biomarkers as defined herein;
(c) comparing the amounts of the analyte biomarkers in the biological sample with the amounts present in a normal control biological sample from a normal subject, such that a difference in the level of the analyte biomarkers in the biological sample is indicative of schizophrenia or other psychotic disorder, or predisposition thereto.

According to a fifth aspect of the invention, there is provided a method of monitoring efficacy of a therapy in a subject having, suspected of having, or of being predisposed to schizophrenia or other psychotic disorder, comprising detecting and/or quantifying, in a sample from said subject, the analyte biomarkers defined herein.

According to a sixth aspect of the invention, there is provided a method of determining the efficacy of therapy for schizophrenia or other psychotic disorder in an individual subject comprising:
(a) obtaining a biological sample from an individual;
(b) quantifying the amounts of the analyte biomarkers as defined herein;
(c) comparing the amounts of the analyte biomarkers in the biological sample with the amounts present in a sample obtained from the individual on a previous occasion, such that a difference in the level of the analyte biomarkers in the biological sample is indicative of a beneficial effect of the therapy.

A further aspect of the invention provides ligands, such as naturally occurring or chemically synthesised compounds, capable of specific binding to the peptide biomarker. A ligand according to the invention may comprise a peptide, an antibody or a fragment thereof, or an aptamer or oligonucleotide, capable of specific binding to the peptide biomarker. The antibody can be a monoclonal antibody or a fragment thereof capable of specific binding to the peptide biomarker. A ligand according to the invention may be labelled with a detectable marker, such as a luminescent, fluorescent or radioactive marker; alternatively or additionally a ligand according to the invention may be labelled with an affinity tag, e.g. a biotin, avidin, streptavidin or His (e.g. hexa-His) tag.

A biosensor according to the invention may comprise the peptide biomarker or a structural/shape mimic thereof capable of specific binding to an antibody against the peptide biomarker. Also provided is an array comprising a ligand or mimic as described herein.

Also provided by the invention is the use of one or more ligands as described herein, which may be naturally occurring or chemically synthesised, and is suitably a peptide, antibody or fragment thereof, aptamer or oligonucleotide, or the use of a biosensor of the invention, or an array of the invention, or a kit of the invention to detect and/or quantify the peptide. In these uses, the detection and/or quantification can be performed on a biological sample such as from the group consisting of CSF, whole blood, blood serum, plasma, urine, saliva, or other bodily fluid, breath, e.g. as condensed breath, or an extract or purification therefrom, or dilution thereof.

Diagnostic or monitoring kits are provided for performing methods of the invention. Such kits will suitably comprise a ligand according to the invention, for detection and/or quantification of the peptide biomarker, and/or a biosensor, and/or an array as described herein, optionally together with instructions for use of the kit.

A further aspect of the invention is a kit for monitoring or diagnosing schizophrenia or other psychotic disorder, comprising a biosensor capable of detecting and/or quantifying the analyte biomarkers as defined herein.

Biomarkers for schizophrenia or other psychotic disorder are essential targets for discovery of novel targets and drug molecules that retard or halt progression of the disorder. As the level of the peptide biomarker is indicative of disorder and of drug response, the biomarker is useful for identification of novel therapeutic compounds in in vitro and/or in vivo assays. Biomarkers of the invention can be employed in methods for screening for compounds that modulate the activity of the peptide.

Thus, in a further aspect of the invention, there is provided the use of a ligand, as described, which can be a peptide, antibody or fragment thereof or aptamer or oligonucleotide according to the invention; or the use of a biosensor according to the invention, or an array according to the invention; or a kit according to the invention, to identify a substance capable of promoting and/or of suppressing the generation of the biomarker.

Also there is provided a method of identifying a substance capable of promoting or suppressing the generation of the peptide in a subject, comprising administering a test substance to a subject animal and detecting and/or quantifying the level of the peptide biomarker present in a test sample from the subject.

### DETAILED DESCRIPTION OF THE INVENTION

According to a first aspect of the invention, there is provided the use of Complement factor H or a functional fragment or phosphorylated fragment thereof, as a biomarker for schizophrenia or other psychotic disorder, or predisposition thereto.

Data is provided herein which demonstrates that the analytes of the invention provide statistically significant biomarkers for the diagnosis of schizophrenia from healthy control patients.

References herein to “functional fragment” include a fragment (e.g. a fragment with C-terminal truncation or with N-terminal truncation) of the peptide which retains the function of providing a biomarker for the diagnosis of schizophrenia or other psychotic disorder. Typically, the fragment will comprise a peptide sequence of greater than 4 amino acids in length, for example 5, 6, 7, 8, 9, 10, 11, 12, 13, 14, 15, 16, 17, 18, 19, or 20 amino acids in length.

In one embodiment, the analyte is suitably Complement factor H or a functional fragment thereof. In a further embodiment, the functional fragment of Complement factor H comprises any of the peptides of SEQ ID NOS: 1 to 3. The peptides of SEQ ID NOS: 1 to 3 were demonstrated in Study 1 herein to be statistically significant between schizophrenia patients and healthy controls.

References herein to “phosphorylated derivative” refer to a peptide which comprises one or more phosphorylated amino acid residues. Typically, the phosphorylated peptide will contain 1, 2 or 3 phosphorylated amino acid residues, such as 1 or 2 phosphorylated amino acid residues. It will be appreciated that such derivatives include fragments of the peptides as defined hereinbefore, i.e. a functional phosphorylated fragment.

In one embodiment, the analyte is suitably Complement factor H or a functional phosphorylated fragment thereof. In one embodiment, the functional phosphorylated fragment of Complement factor H is selected from any of the phosphopeptides of SEQ ID NOS: 15 and 53 (i.e. wherein the Thr at position 3 and the Cys at position 4 of SEQ ID NO: 15 are both phosphorylated as described in Table 7; and wherein the Thr at position 13 and the Ser at position 17 of SEQ ID NO: 53 are both phosphorylated as described in Table 16). The phosphopeptides of SEQ ID NOS: 15 and 53 are demonstrated in Studies 1 and 2 herein to be statistically significant between schizophrenia patients and healthy controls.

In one embodiment of the invention, the use additionally comprises one or more additional analytes selected from: Condensin complex subunit 2, DNA-directed RNA polymerase III subunit RPC5, Daple, Coagulation factor XIII B chain, B box and SPRY domain-containing protein, Synaptotagmin-2, Zinc-alpha-2-glycoprotein, Uncharacterised protein C1orf125, Synemin, Collagen alpha-1(V) chain, Leucine-rich repeat-containing protein 16A, Vang-like protein 1, Alpha-actinin-2, Probable ATP-dependent DNA helicase HFM1, Fibulin-1, DNA-directed RNA polymerase III subunit RPC1, RANBP2-like and GRIP domain-containing protein 1, Putative zinc-alpha-2-glycoprotein-like 1, Alpha-2-antiplasmin, Dedicator of cytokinesis protein 3, Phosphorylated CTD-interacting factor 1, Inactive ubiquitin carboxyl-terminal hydrolase 54, Serine/threonine protein kinase 38-like, Ig kappa chain V-I region WEA, Ig kappa chain V-III region SIE, Protein GREB1, Seprase, Pre-mRNA-processing factor 6, Spermatogenesis-associated protein 21 (SPAT21), Protein SOLO, Apolipoprotein F, Apolipoprotein M, Kininogen-1 (KNG1), Complement component 8 gamma chain (CO8G), NACHT and WD repeat domain-containing protein 1 (NWD1), Annexin A6 (ANXA6), Ig heavy chain V-III region BRO, Complement factor H-related protein 2, Prothrombin Thrombin heavy chain, Complement factor H, Histidine-rich glycoprotein, Ig gamma-2 chain C region, Mitochondrial dicarboxylate protein, CD5 ligand, Transferrin, Apolipoprotein D, Apolipoprotein A2, Apolipoprotein A4, Ig mu chain C region (IgM), Leucine zipper-EF-hand-containing transmembrane protein 1, CD5 antigen-like (SP-alpha), Alpha-1-antitrypsin-related protein, Apolipoprotein A1, Serotransferrin, Apolipoprotein C1, Alpha-2-HS-glycoprotein (fetuin A), Hemopexin precursor (Beta-1B-glycoprotein), Apolipoprotein C-III, Apolipoprotein E, Inter-alpha-trypsin inhibitor heavy chain H4, Inter-alpha-trypsin inhibitor heavy chain H3, Myosin-XVIIIa, Complement C4-A, Lumican, Microtubule-associated protein 2, Apolipoprotein L1, Haptoglobin, Gelsolin, CDK5 regulatory subunit-associated protein 2, Retinol-binding protein 4 (RBP4), Myoferlin, Peroxisome proliferator-activated receptor gamma coactivator-related protein 1, Neurexin-1-alpha, Prostate-specific membrane antigen-like protein, Creatine kinase, Vitamin K-dependent protein S precursor, Inter-alpha-trypsin inhibitor heavy chain H2, C4b-binding protein alpha chain, Complement component C7 and Beta-2-glycoprotein 1 (Apolipoprotein H).

According to one particular aspect of the invention, there is provided the use of one or more analytes selected from: Condensin complex subunit 2, DNA-directed RNA polymerase III subunit RPC5, Daple, Coagulation factor XIII B chain, B box and SPRY domain-containing protein, Synaptotagmin-2, Zinc-alpha-2-glycoprotein, Uncharacterised protein C1orf125, Synemin, Collagen alpha-1(V) chain, Leucine-rich repeat-containing protein 16A, Vang-like protein 1, Alpha-actinin-2, Probable ATP-dependent DNA helicase HFM1, Fibulin-1, DNA-directed RNA polymerase III subunit RPC1, RANBP2-like and GRIP domain-containing protein 1, Putative zinc-alpha-2-glycoprotein-like 1, Alpha-2-antiplasmin, Dedicator of cytokinesis protein 3, Phosphorylated CTD-interacting factor 1, Inactive ubiquitin carboxyl-terminal hydrolase 54, Serine/threonine protein kinase 38-like, Ig kappa chain V-I region WEA, Ig kappa chain V-III region SIE, Protein GREB1, Seprase, Pre-mRNA-processing factor 6, Spermatogenesis-associated protein 21 (SPAT21), Protein SOLO, Apolipoprotein F, Apolipoprotein M, Kininogen-1 (KNG1), Complement component 8 gamma chain (CO8G), NACHT and WD repeat domain-containing protein 1 (NWD1), Annexin A6 (ANXA6), Ig heavy chain V-III region BRO, Complement factor H-related protein 2, Prothrombin Thrombin heavy chain, Complement factor H, Histidine-rich glycoprotein, Ig gamma-2 chain C region and Mitochondrial dicarboxylate protein, or a functional fragment or phosphorylated derivative thereof, as a biomarker for schizophrenia or other psychotic disorder, or predisposition thereto.

In one embodiment of the invention, the analyte is selected from Condensin complex subunit 2, DNA-directed RNA polymerase III subunit RPC5, Daple, Coagulation factor XIII B chain, B box and SPRY domain-containing protein, Synemin, Collagen alpha-1(V) chain, Leucine-rich repeat-containing protein 16A, Vang-like protein 1, Alpha-actinin-2, Probable ATP-dependent DNA helicase HFM1, Fibulin-1, DNA-directed RNA polymerase III subunit RPC1, RANBP2-like and GRIP domain-containing protein 1, Putative zinc-alpha-2-glycoprotein-like 1, Alpha-2-antiplasmin, Dedicator of cytokinesis protein 3, Phosphorylated CTD-interacting factor 1, Inactive ubiquitin carboxyl-terminal hydrolase 54, Serine/threonine protein kinase 38-like, Ig kappa chain V-I region WEA, Mitochondrial dicarboxylate protein, Ig kappa chain V-III region SIE, Protein GREB1, Seprase, Pre-mRNA-processing factor 6, Spermatogenesis-associated protein 21 (SPAT21), Protein SOLO and Apolipoprotein F. The analytes of this embodiment were found to be statistically significant at the protein level between schizophrenia patients and healthy controls in accordance with the results shown in Studies 1 and 2 herein.

In an alternative embodiment of the invention, the analyte is selected from: DNA-directed RNA polymerase III subunit RPC5, Complement factor H, Synaptotagmin-2, Apolipoprotein M, Kininogen-1 (KNG1), Complement component 8 gamma chain (CO8G), NACHT and WD repeat domain-containing protein 1 (NWD1) and Annexin A6 (ANXA6). The analytes of this embodiment were found to be statistically significant at the peptide level between schizophrenia patients and healthy controls in accordance with the results shown in Studies 1 and 2 herein. In a further embodiment of the first aspect of the invention, the analyte is selected from: DNA-directed RNA polymerase III subunit RPC5, Complement factor H, Synaptotagmin-2, Apolipoprotein M, Kininogen-1 (KNG1), Complement component 8 gamma chain (CO8G), NACHT and WD repeat domain-containing protein 1 (NWD1) and Annexin A6 (ANXA6) or a functional fragment thereof.

When the analyte represents DNA-directed RNA polymerase III subunit RPC5, said analyte is suitably DNA-directed RNA polymerase III subunit RPC5 or a functional fragment thereof. In one embodiment, the functional fragment of DNA-directed RNA polymerase III subunit RPC5 comprises the peptides of SEQ ID NOS: 10 and 11. In a further embodiment, the functional fragment of DNA-directed RNA polymerase III subunit RPC5 comprises the peptide of SEQ ID NO: 10. The peptide of SEQ ID NO: 10 is demonstrated in Study 1 herein to be statistically significant between schizophrenia patients and healthy controls.

When the analyte represents Synaptotagmin-2, said analyte is suitably Synaptotagmin-2 or a functional fragment thereof. In one embodiment, the functional fragment of Synaptotagmin-2 comprises the peptides of SEQ ID NOS: 12 and 13. The peptides of SEQ ID NOS: 12 and 13 are demonstrated in Study 1 herein to be statistically significant between schizophrenia patients and healthy controls.

When the analyte represents Apolipoprotein M, said analyte is suitably Apolipoprotein M or a functional fragment thereof. In one embodiment, the functional fragment of Apolipoprotein M comprises the peptide of SEQ ID NO: 26. The peptide of SEQ ID NO: 26 is demonstrated in Study 2 herein to be statistically significant between schizophrenia patients and healthy controls.

When the analyte represents Kininogen-1 (KNG1), said analyte is suitably Kininogen-1 (KNG1) or a functional fragment thereof. In one embodiment, the functional fragment of Kininogen-1 (KNG1) comprises the peptide of SEQ ID NO: 27. The peptide of SEQ ID NO: 27 is demonstrated in Study 2 herein to be statistically significant between schizophrenia patients and healthy controls.

When the analyte represents Complement component 8 gamma chain (CO8G), said analyte is suitably Complement component 8 gamma chain (CO8G) or a functional fragment thereof. In one embodiment, the functional fragment of Complement component 8 gamma chain (CO8G) comprises the peptide of SEQ ID NOS: 32 to 34. The peptides of SEQ ID NOS: 32 to 34 are demonstrated in Study 2 herein to be statistically significant between schizophrenia patients and healthy controls.

When the analyte represents NACHT and WD repeat domain-containing protein 1 (NWD1), said analyte is suitably NACHT and WD repeat domain-containing protein 1 (NWD1) or a functional fragment thereof. In one embodiment, the functional fragment of NACHT and WD repeat domain-containing protein 1 (NWD1) comprises the peptide of SEQ ID NO: 35. The peptide of SEQ ID NO: 35 is demonstrated in Study 2 herein to be statistically significant between schizophrenia patients and healthy controls.

When the analyte represents Annexin A6 (ANXA6), said analyte is suitably Annexin A6 (ANXA6) or a functional fragment thereof. In one embodiment, the functional fragment of Annexin A6 (ANXA6) comprises the peptide of SEQ ID NOS: 36 and 37. The peptides of SEQ ID NO: 36 and 37 are demonstrated in Study 2 herein to be statistically significant between schizophrenia patients and healthy controls.

In an alternative embodiment of the invention, the analyte is selected from: Complement factor H, Ig gamma-2 chain C region, Zinc-alpha-2-glycoprotein, Uncharacterised protein C1orf125, Ig heavy chain V-III region BRO, Complement factor H-related protein 2, Prothrombin Thrombin heavy chain and Histidine-rich glycoprotein. The analytes of this embodiment were found to be statistically significant at the phosphopeptide level between schizophrenia patients and healthy controls in accordance with the results shown in Studies 1 and 2 herein. In a further embodiment of the first aspect of the invention, the analyte is selected from: Complement factor H, Ig gamma-2 chain C region, Zinc-alpha-2-glycoprotein, Uncharacterised protein C1orf125, Ig heavy chain V-III region BRO, Complement factor H-related protein 2, Prothrombin Thrombin heavy chain and Histidine-rich glycoprotein or a phosphorylated derivative thereof.

When the analyte represents Ig gamma-2 chain C region, said analyte is suitably Ig gamma-2 chain C region or a functional phosphorylated fragment thereof. In one embodiment, the functional phosphorylated fragment of Ig gamma-2 chain C region comprises the phosphopeptide of SEQ ID NO: 18 (i.e. wherein the Thr at position 11 and the Cys at position 12 of SEQ ID NO: 18 are both phosphorylated as described in Table 7). The phosphopeptide of SEQ ID NO: 18 is demonstrated in Study 1 herein to be statistically significant between schizophrenia patients and healthy controls.

When the analyte represents Zinc-alpha-2-glycoprotein, said analyte is suitably Zinc-alpha-2-glycoprotein or a functional phosphorylated fragment thereof. In one embodiment, the functional phosphorylated fragment of Zinc-alpha-2-glycoprotein comprises the phosphopeptide of SEQ ID NO: 20 (i.e. wherein the Tyr at position 7 of SEQ ID NO: 20 is phosphorylated as described in Table 7). The phosphopeptide of SEQ ID NO: 20 is demonstrated in Study 1 herein to be statistically significant between schizophrenia patients and healthy controls.

When the analyte represents Uncharacterised protein C1orf125, said analyte is suitably Uncharacterised protein C1orf125 or a functional phosphorylated fragment thereof. In one embodiment, the functional phosphorylated fragment of Uncharacterised protein C1orf125 comprises the phosphopeptide of SEQ ID NO: 24 (i.e. wherein the Tyr at position 1, the Ser at position 8 and the Thr at position 10 of SEQ ID NO: 24 is phosphorylated as described in Table 7). The phosphopeptide of SEQ ID NO: 24 is demonstrated in Study 1 herein to be statistically significant between schizophrenia patients and healthy controls.

When the analyte represents Ig heavy chain V-III region BRO, said analyte is suitably Ig heavy chain V-III region BRO or a functional phosphorylated fragment thereof. In one embodiment, the functional phosphorylated fragment of Ig heavy chain V-III region BRO comprises the phosphopeptide of SEQ ID NO: 47 (i.e. wherein the Thr at position 10 is phosphorylated as described in Table 16). The phosphopeptide of SEQ ID NO: 47 is demonstrated in Study 2 herein to be statistically significant between schizophrenia patients and healthy controls.

When the analyte represents Complement factor H-related protein 2, said analyte is suitably Complement factor H-related protein 2 or a functional phosphorylated fragment thereof. In one embodiment, the functional phosphorylated fragment of Complement factor H-related protein 2 comprises the phosphopeptide of SEQ ID NO: 48 (i.e. wherein the Ser at position 16 is phosphorylated as described in Table 16). The phosphopeptide of SEQ ID NO: 48 is demonstrated in Study 2 herein to be statistically significant between schizophrenia patients and healthy controls.

When the analyte represents Prothrombin Thrombin heavy chain, said analyte is suitably Prothrombin Thrombin heavy chain or a functional phosphorylated fragment thereof. In one embodiment, the functional phosphorylated fragment of Prothrombin Thrombin heavy chain comprises the phosphopeptide of SEQ ID NO: 50 (i.e. wherein the Ser at position 2 and the Tyr at position 8 is phosphorylated as described in Table 16). The phosphopeptide of SEQ ID NO: 50 is demonstrated in Study 2 herein to be statistically significant between schizophrenia patients and healthy controls.

When the analyte represents Histidine-rich glycoprotein, said analyte is suitably Histidine-rich glycoprotein or a functional phosphorylated fragment thereof. In one embodiment, the functional phosphorylated fragment of Histidine-rich glycoprotein comprises the phosphopeptide of SEQ ID NO: 54 (i.e. wherein the Tyr at position 14 and the Ser at position 23 is phosphorylated as described in Table 16). The phosphopeptide of SEQ ID NO: 54 is demonstrated in Study 2 herein to be statistically significant between schizophrenia patients and healthy controls.

In one embodiment of the invention, the analyte is other than Coagulation factor XIII B chain, Synaptotagmin-2, Inactive ubiquitin carboxyl-terminal hydrolase 54, Apolipoprotein F and Apolipoprotein M.

Thus, according to a further aspect of the invention, there is provided the use of one or more analytes selected from: Condensin complex subunit 2, DNA-directed RNA polymerase III subunit RPC5, Daple, B box and SPRY domain-containing protein, Zinc-alpha-2-glycoprotein, Uncharacterised protein C1orf125, Synemin, Collagen alpha-1(V) chain, Leucine-rich repeat-containing protein 16A, Vang-like protein 1, Alpha-actinin-2, Probable ATP-dependent DNA helicase HFM1, Fibulin-1, DNA-directed RNA polymerase III subunit RPC1, RANBP2-like and GRIP domain-containing protein 1, Putative zinc-alpha-2-glycoprotein-like 1, Alpha-2-antiplasmin, Dedicator of cytokinesis protein 3, Phosphorylated CTD-interacting factor 1, Serine/threonine protein kinase 38-like, Ig kappa chain V-I region WEA, Ig kappa chain V-III region SIE, Protein GREB1, Seprase, Pre-mRNA-processing factor 6, Spermatogenesis-associated protein 21 (SPAT21), Protein SOLO, Kininogen-1 (KNG1), Complement component 8 gamma chain (CO8G), NACHT and WD repeat domain-containing protein 1 (NWD1), Annexin A6 (ANXA6), Ig heavy chain V-III region BRO, Complement factor H-related protein 2, Prothrombin Thrombin heavy chain, Complement factor H, Histidine-rich glycoprotein, Ig gamma-2 chain C region and Mitochondrial dicarboxylate protein, as a biomarker for schizophrenia or other psychotic disorder, or predisposition thereto.

In one embodiment, the use comprises any of the peptides selected from SEQ ID NOs 1 to 54, or a phosphorylated derivative thereof.

According to a second aspect of the invention, there is provided the use of one or more peptides selected from SEQ ID NOs 1 to 54, or a phosphorylated derivative thereof, as a biomarker for schizophrenia or other psychotic disorder, or predisposition thereto.

According to a further aspect of the invention, there is provided the use of Complement factor H, Condensin complex subunit 2, DNA-directed RNA polymerase III subunit RPC5, Daple, Coagulation factor XIII B chain, B box and SPRY domain-containing protein, Synaptotagmin-2, Zinc-alpha-2-glycoprotein, Uncharacterised protein C1orf125, Synemin, Collagen alpha-1(V) chain, Leucine-rich repeat-containing protein 16A, Vang-like protein 1, Alpha-actinin-2, Probable ATP-dependent DNA helicase HFM1, Fibulin-1, DNA-directed RNA polymerase III subunit RPC1, RANBP2-like and GRIP domain-containing protein 1, Putative zinc-alpha-2-glycoprotein-like 1, Alpha-2-antiplasmin, Dedicator of cytokinesis protein 3, Phosphorylated CTD-interacting factor 1, Inactive ubiquitin carboxyl-terminal hydrolase 54, Serine/threonine protein kinase 38-like, Ig kappa chain V-I region WEA, Ig kappa chain V-III region SIE, Protein GREB1, Seprase, Pre-mRNA-processing factor 6, Spermatogenesis-associated protein 21 (SPAT21), Protein SOLO, Apolipoprotein F, Apolipoprotein M, Kininogen-1 (KNG1), Complement component 8 gamma chain (CO8G), NACHT and WD repeat domain-containing protein 1 (NWD1), Annexin A6 (ANXA6), Ig heavy chain V-III region BRO, Complement factor H-related protein 2, Prothrombin Thrombin heavy chain, Histidine-rich glycoprotein, Ig gamma-2 chain C region, Mitochondrial dicarboxylate protein, CD5 ligand, Transferrin, Apolipoprotein D, Apolipoprotein A2, Apolipoprotein A4, Ig mu chain C region (IgM), Leucine zipper-EF-hand-containing transmembrane protein 1, CD5 antigen-like (SP-alpha), Alpha-1-antitrypsin-related protein, Apolipoprotein A1, Serotransferrin, Apolipoprotein C1, Alpha-2-HS-glycoprotein (fetuin A), Hemopexin precursor (Beta-1B-glycoprotein), Apolipoprotein C-III, Apolipoprotein E, Inter-alpha-trypsin inhibitor heavy chain H4, Inter-alpha-trypsin inhibitor heavy chain H3, Myosin-XVIIIa, Complement C4-A, Lumican, Microtubule-associated protein 2, Apolipoprotein L1, Haptoglobin, Gelsolin, CDK5 regulatory subunit-associated protein 2, Retinol-binding protein 4 (RBP4), Myoferlin, Peroxisome proliferator-activated receptor gamma coactivator-related protein 1, Neurexin-1-alpha, Prostate-specific membrane antigen-like protein, Creatine kinase, Vitamin K-dependent protein S precursor, Inter-alpha-trypsin inhibitor heavy chain H2, C4b-binding protein alpha chain, Complement component C7 and Beta-2-glycoprotein 1 (Apolipoprotein H), as a specific panel of analyte biomarkers for schizophrenia or other psychotic disorder, or predisposition thereto.

The term “biomarker” means a distinctive biological or biologically derived indicator of a process, event, or condition. Peptide biomarkers can be used in methods of diagnosis, e.g. clinical screening, and prognosis assessment and in monitoring the results of therapy, identifying patients most likely to respond to a particular therapeutic treatment, drug screening and development. Biomarkers and uses thereof are valuable for identification of new drug treatments and for discovery of new targets for drug treatment.

As used herein, the term “biosensor” means anything capable of detecting the presence of the biomarker. Examples of biosensors are described herein.

References herein to “other psychotic disorder” relate to any appropriate psychotic disorder according to DSM-IV Diagnostic and Statistical Manual of Mental Disorders, 4th edition, American Psychiatric Assoc, Washington, D.C., 2000. In one particular embodiment, the other psychotic disorder is a psychotic disorder related to schizophrenia. Examples of psychotic disorders related to schizophrenia include brief psychotic disorder delusional disorder, psychotic disorder due to a general medical condition, schizoeffective disorder, schizophreniform disorder, and substance-induced psychotic disorder.

In one embodiment, one or more of the biomarkers defined hereinbefore may be replaced by a molecule, or a measurable fragment of the molecule, found upstream or downstream of the biomarker in a biological pathway.

Biosensors according to the invention may comprise a ligand or ligands, as described herein, capable of specific binding to the peptide biomarker. Such biosensors are useful in detecting and/or quantifying a peptide of the invention.

Diagnostic kits for the diagnosis and monitoring of schizophrenia or other psychotic disorder are described herein. In one embodiment, the kits additionally contain a biosensor capable of detecting and/or quantifying a peptide biomarker.

Monitoring methods of the invention can be used to monitor onset, progression, stabilisation, amelioration and/or remission.

In methods of diagnosing or monitoring according to the invention, detecting and/or quantifying the peptide biomarker in a biological sample from a test subject may be performed on two or more occasions. Comparisons may be made between the level of biomarker in samples taken on two or more occasions. Assessment of any change in the level of the peptide biomarker in samples taken on two or more occasions may be performed. Modulation of the peptide biomarker level is useful as an indicator of the state of schizophrenia or other psychotic disorder or predisposition thereto. An increase in the level of the biomarker, over time is indicative of onset or progression, i.e. worsening of this disorder, whereas a decrease in the level of the peptide biomarker indicates amelioration or remission of the disorder, or vice versa.

A method of diagnosis of or monitoring according to the invention may comprise quantifying the peptide biomarker in a test biological sample from a test subject and comparing the level of the peptide present in said test sample with one or more controls.

The control used in a method of the invention can be one or more control(s) selected from the group consisting of: the level of biomarker peptide found in a normal control sample from a normal subject, a normal biomarker peptide level; a normal biomarker peptide range, the level in a sample from a subject with schizophrenia or other psychotic disorder, or a diagnosed predisposition thereto; schizophrenia or other psychotic disorder biomarker peptide level, or schizophrenia or other psychotic disorder biomarker peptide range.

In one embodiment, there is provided a method of diagnosing schizophrenia or other psychotic disorder, or predisposition thereto, which comprises:
(a) quantifying the amount of the peptide biomarker in a test biological sample; and
(b) comparing the amount of said peptide in said test sample with the amount present in a normal control biological sample from a normal subject.

For biomarkers which are increased in patients with schizophrenia or other psychotic disorder, a higher level of the peptide biomarker in the test sample relative to the level in the normal control is indicative of the presence of schizophrenia or other psychotic disorder, or predisposition thereto; an equivalent or lower level of the peptide in the test sample relative to the normal control is indicative of absence of schizophrenia or other psychotic disorder and/or absence of a predisposition thereto. For biomarkers which are decreased in patients with schizophrenia or other psychotic disorder, a lower level of the peptide biomarker in the test sample relative to the level in the normal control is indicative of the presence of schizophrenia or other psychotic disorder, or predisposition thereto; an equivalent or lower level of the peptide in the test sample relative to the normal control is indicative of absence of schizophrenia or other psychotic disorder and/or absence of a predisposition thereto.

The term “diagnosis” as used herein encompasses identification, confirmation, and/or characterisation of schizophrenia or other psychotic disorder, or predisposition thereto. By predisposition it is meant that a subject does not currently present with the disorder, but is liable to be affected by the disorder in time. Methods of monitoring and of diagnosis according to the invention are useful to confirm the existence of a disorder, or predisposition thereto; to monitor development of the disorder by assessing onset and progression, or to assess amelioration or regression of the disorder. Methods of monitoring and of diagnosis are also useful in methods for assessment of clinical screening, prognosis, choice of therapy, evaluation of therapeutic benefit, i.e. for drug screening and drug development.

Efficient diagnosis and monitoring methods provide very powerful “patient solutions” with the potential for improved prognosis, by establishing the correct diagnosis, allowing rapid identification of the most appropriate treatment (thus lessening unnecessary exposure to harmful drug side effects), reducing “down-time” and relapse rates.

Also provided is a method of monitoring efficacy of a therapy for schizophrenia or other psychotic disorder in a subject having such a disorder, suspected of having such a disorder, or of being predisposed thereto, comprising detecting and/or quantifying the peptide present in a biological sample from said subject. In monitoring methods, test samples may be taken on two or more occasions. The method may further comprise comparing the level of the biomarker(s) present in the test sample with one or more control(s) and/or with one or more previous test sample(s) taken earlier from the same test subject, e.g. prior to commencement of therapy, and/or from the same test subject at an earlier stage of therapy. The method may comprise detecting a change in the level of the biomarker(s) in test samples taken on different occasions.

The invention provides a method for monitoring efficacy of therapy for schizophrenia or other psychotic disorder in a subject, comprising:
(a) quantifying the amount of the peptide biomarker; and
(b) comparing the amount of said peptide in said test sample with the amount present in one or more control(s) and/or one or more previous test sample(s) taken at an earlier time from the same test subject.

For biomarkers which are increased in patients with schizophrenia or other psychotic disorder, a decrease in the level of the peptide biomarker in the test sample relative to the level in a previous test sample taken earlier from the same test subject is indicative of a beneficial effect, e.g. stabilisation or improvement, of said therapy on the disorder, suspected disorder or predisposition thereto. For biomarkers which are decreased in patients with schizophrenia or other psychotic disorder, an increase in the level of the peptide biomarker in the test sample relative to the level in a previous test sample taken earlier from the same test subject is indicative of a beneficial effect, e.g. stabilisation or improvement, of said therapy on the disorder, suspected disorder or predisposition thereto.

Methods for monitoring efficacy of a therapy can be used to monitor the therapeutic effectiveness of existing therapies and new therapies in human subjects and in non-human animals (e.g. in animal models). These monitoring methods can be incorporated into screens for new drug substances and combinations of substances.

Suitably, the time elapsed between taking samples from a subject undergoing diagnosis or monitoring will be 3 days, 5 days, a week, two weeks, a month, 2 months, 3 months, 6 or 12 months. Samples may be taken prior to and/or during and/or following an anti-psychotic therapy. Samples can be taken at intervals over the remaining life, or a part thereof, of a subject.

The term “detecting” as used herein means confirming the presence of the peptide biomarker present in the sample. Quantifying the amount of the biomarker present in a sample may include determining the concentration of the peptide biomarker present in the sample. Detecting and/or quantifying may be performed directly on the sample, or indirectly on an extract therefrom, or on a dilution thereof.

In alternative aspects of the invention, the presence of the peptide biomarker is assessed by detecting and/or quantifying antibody or fragments thereof capable of specific binding to the biomarker that are generated by the subject's body in response to the peptide and thus are present in a biological sample from a subject having schizophrenia or other psychotic disorder or a predisposition thereto.

Detecting and/or quantifying can be performed by any method suitable to identify the presence and/or amount of a specific protein in a biological sample from a patient or a purification or extract of a biological sample or a dilution thereof. In methods of the invention, quantifying may be performed by measuring the concentration of the peptide biomarker in the sample or samples. Biological samples that may be tested in a method of the invention include cerebrospinal fluid (CSF), whole blood, blood serum, plasma, urine, saliva, or other bodily fluid (stool, tear fluid, synovial fluid, sputum), breath, e.g. as condensed breath, or an extract or purification therefrom, or dilution thereof. Biological samples also include tissue homogenates, tissue sections and biopsy specimens from a live subject, or taken post-mortem. The samples can be prepared, for example where appropriate diluted or concentrated, and stored in the usual manner.

The biomarker may be directly detected, e.g. by SELDI or MALDI-TOF. Alternatively, the biomarker may be detected directly or indirectly via interaction with a ligand or ligands such as an antibody or a biomarker-binding fragment thereof, or other peptide, or ligand, e.g. aptamer, or oligonucleotide, capable of specifically binding the biomarker. The ligand may possess a detectable label, such as a luminescent, fluorescent or radioactive label, and/or an affinity tag.

For example, detecting and/or quantifying can be performed by one or more method(s) selected from the group consisting of: SELDI (-TOF), MALDI (-TOF), a 1-D gel-based analysis, a 2-D gel-based analysis, Mass spec (MS), reverse phase (RP) LC, size permeation (gel filtration), ion exchange, affinity, HPLC, UPLC and other LC or LC MS-based techniques. Appropriate LC MS techniques include ICAT® (Applied Biosystems, CA, USA), or iTRAQ® (Applied Biosystems, CA, USA). Liquid chromatography (e.g. high pressure liquid chromatography (HPLC) or low pressure liquid chromatography (LPLC)), thin-layer chromatography, NMR (nuclear magnetic resonance) spectroscopy could also be used.

Methods of diagnosing or monitoring according to the invention may comprise analysing a sample of cerebrospinal fluid (CSF) by SELDI TOF or MALDI TOF to detect the presence or level of the peptide biomarker. These methods are also suitable for clinical screening, prognosis, monitoring the results of therapy, identifying patients most likely to respond to a particular therapeutic treatment, for drug screening and development, and identification of new targets for drug treatment.

Detecting and/or quantifying the peptide biomarkers may be performed using an immunological method, involving an antibody, or a fragment thereof capable of specific binding to the peptide biomarker. Suitable immunological methods include sandwich immunoassays, such as sandwich ELISA, in which the detection of the peptide biomarkers is performed using two antibodies which recognize different epitopes on a peptide biomarker; radioimmunoassays (RIA), direct, indirect or competitive enzyme linked immunosorbent assays (ELISA), enzyme immunoassays (EIA), Fluorescence immunoassays (FIA), western blotting, immunoprecipitation and any particle-based immunoassay (e.g. using gold, silver, or latex particles, magnetic particles, or Q-dots). Immunological methods may be performed, for example, in microtitre plate or strip format.

Immunological methods in accordance with the invention may be based, for example, on any of the following methods.

Immunoprecipitation is the simplest immunoassay method; this measures the quantity of precipitate, which forms after the reagent antibody has incubated with the sample and reacted with the target antigen present therein to form an insoluble aggregate. Immunoprecipitation reactions may be qualitative or quantitative.

In particle immunoassays, several antibodies are linked to the particle, and the particle is able to bind many antigen molecules simultaneously. This greatly accelerates the speed of the visible reaction. This allows rapid and sensitive detection of the biomarker.

In immunonephelometry, the interaction of an antibody and target antigen on the biomarker results in the formation of immune complexes that are too small to precipitate. However, these complexes will scatter incident light and this can be measured using a nephelometer. The antigen, i.e. biomarker, concentration can be determined within minutes of the reaction.

Radioimmunoassay (RIA) methods employ radioactive isotopes such as I¹²⁵ to label either the antigen or antibody. The isotope used emits gamma rays, which are usually measured following removal of unbound (free) radiolabel. The major advantages of RIA, compared with other immunoassays, are higher sensitivity, easy signal detection, and well-established, rapid assays. The major is disadvantages are the health and safety risks posed by the use of radiation and the time and expense associated with maintaining a licensed radiation safety and disposal program. For this reason, RIA has been largely replaced in routine clinical laboratory practice by enzyme immunoassays.

Enzyme (EIA) immunoassays were developed as an alternative to radioimmunoassays (RIA). These methods use an enzyme to label either the antibody or target antigen. The sensitivity of EIA approaches that for RIA, without the danger posed by radioactive isotopes. One of the most widely used EIA methods for detection is the enzyme-linked immunosorbent assay (ELISA). ELISA methods may use two antibodies one of which is specific for the target antigen and the other of which is coupled to an enzyme, addition of the substrate for the enzyme results in production of a chemiluminescent or fluorescent signal.

Fluorescent immunoassay (FIA) refers to immunoassays which utilize a fluorescent label or an enzyme label which acts on the substrate to form a fluorescent product. Fluorescent measurements are inherently more sensitive than colorimetric (spectrophotometric) measurements. Therefore, FIA methods have greater analytical sensitivity than EIA methods, which employ absorbance (optical density) measurement.

Chemiluminescent immunoassays utilize a chemiluminescent label, which produces light when excited by chemical energy; the emissions are measured using a light detector.

Immunological methods according to the invention can thus be performed using well-known methods. Any direct (e.g., using a sensor chip) or indirect procedure may be used in the detection of peptide biomarkers of the invention.

The Biotin-Avidin or Biotin-Streptavidin systems are generic labelling systems that can be adapted for use in immunological methods of the invention. One binding partner (hapten, antigen, ligand, aptamer, antibody, enzyme etc) is labelled with biotin and the other partner (surface, e.g. well, bead, sensor etc) is labelled with avidin or streptavidin. This is conventional technology for immunoassays, gene probe assays and (bio)sensors, but is an indirect immobilisation route rather than a direct one. For example a biotinylated ligand (e.g. antibody or aptamer) specific for a peptide biomarker of the invention may be immobilised on an avidin or streptavidin surface, the immobilised ligand may then be exposed to a sample containing or suspected of containing the peptide biomarker in order to detect and/or quantify a peptide biomarker of the invention. Detection and/or quantification of the immobilised antigen may then be performed by an immunological method as described herein.

The term “antibody” as used herein includes, but is not limited to: polyclonal, monoclonal, bispecific, humanised or chimeric antibodies, single chain antibodies, Fab fragments and F(ab′)₂ fragments, fragments produced by a Fab expression library, anti-idiotypic (anti-Id) antibodies and epitope-binding fragments of any of the above. The term “antibody” as used herein also refers to immunoglobulin molecules and immunologically-active portions of immunoglobulin molecules, i.e., molecules that contain an antigen binding site that specifically binds an antigen. The immunoglobulin molecules of the invention can be of any class (e.g., IgG, IgE, IgM, IgD and IgA) or subclass of immunoglobulin molecule.

The identification of key biomarkers specific to a disease is central to integration of diagnostic procedures and therapeutic regimes. Using predictive biomarkers appropriate diagnostic tools such as biosensors can be developed, accordingly, in methods and uses of the invention, detecting and quantifying can be performed using a biosensor, microanalytical system, microengineered system, microseparation system, immunochromatography system or other suitable analytical devices. The biosensor may incorporate an immunological method for detection of the biomarker(s), electrical, thermal, magnetic, optical (e.g. hologram) or acoustic technologies. Using such biosensors, it is possible to detect the target biomarker(s) at the anticipated concentrations found in biological samples.

Thus, according to a further aspect of the invention there is provided an apparatus for diagnosing or monitoring schizophrenia or other psychotic disorder which comprises a biosensor, microanalytical, microengineered, microseparation and/or immunochromatography system configured to detect and/or quantify any of the biomarkers defined herein.

The biomarker(s) of the invention can be detected using a biosensor incorporating technologies based on “smart” holograms, or high frequency acoustic systems, such systems are particularly amenable to “bar code” or array configurations.

In smart hologram sensors (Smart Holograms Ltd, Cambridge, UK), a holographic image is stored in a thin polymer film that is sensitised to react specifically with the biomarker. On exposure, the biomarker reacts with the polymer leading to an alteration in the image displayed by the hologram. The test result read-out can be a change in the optical brightness, image, colour and/or position of the image. For qualitative and semi-quantitative applications, a sensor hologram can be read by eye, thus removing the need for detection equipment. A simple colour sensor can be used to read the signal when quantitative measurements are required. Opacity or colour of the sample does not interfere with operation of the sensor. The format of the sensor allows multiplexing for simultaneous detection of several substances. Reversible and irreversible sensors can be designed to meet different requirements, and continuous monitoring of a particular biomarker of interest is feasible.

Suitably, biosensors for detection of one or more biomarkers of the invention combine biomolecular recognition with appropriate means to convert detection of the presence, or quantitation, of the biomarker in the sample into a signal. Biosensors can be adapted for “alternate site” diagnostic testing, e.g. in the ward, outpatients' department, surgery, home, field and workplace.

Biosensors to detect one or more biomarkers of the invention include acoustic, plasmon resonance, holographic and microengineered sensors. Imprinted recognition elements, thin film transistor technology, magnetic acoustic resonator devices and other novel acousto-electrical systems may be employed in biosensors for detection of the one or more biomarkers of the invention.

Methods involving detection and/or quantification of one or more peptide biomarkers of the invention can be performed on bench-top instruments, or can be incorporated onto disposable, diagnostic or monitoring platforms that can be used in a non-laboratory environment, e.g. in the physician's office or at the patient's bedside. Suitable biosensors for performing methods of the invention include “credit” cards with optical or acoustic readers. Biosensors can be configured to allow the data collected to be electronically transmitted to the physician for interpretation and thus can form the basis for e-neuromedicine.

Any suitable animal may be used as a subject non-human animal, for example a non-human primate, horse, cow, pig, goat, sheep, dog, cat, fish, rodent, e.g. guinea pig, rat or mouse; insect (e.g. Drosophila), amphibian (e.g. Xenopus) or C. elegans.

The test substance can be a known chemical or pharmaceutical substance, such as, but not limited to, an anti-psychotic disorder therapeutic; or the test substance can be novel synthetic or natural chemical entity, or a combination of two or more of the aforesaid substances.

There is provided a method of identifying a substance capable of promoting or suppressing the generation of the peptide biomarker in a subject, comprising exposing a test cell to a test substance and monitoring the level of the peptide biomarker within said test cell, or secreted by said test cell.

The test cell could be prokaryotic, however a eukaryotic cell will suitably be employed in cell-based testing methods. Suitably, the eukaryotic cell is a yeast cell, insect cell, Drosophila cell, amphibian cell (e.g. from Xenopus), C. elegans cell or is a cell of human, non-human primate, equine, bovine, porcine, caprine, ovine, canine, feline, piscine, rodent or murine origin.

In methods for identifying substances of potential therapeutic use, non-human animals or cells can be used that are capable of expressing the peptide.

Screening methods also encompass a method of identifying a ligand capable of binding to the peptide biomarker according to the invention, comprising incubating a test substance in the presence of the peptide biomarker in conditions appropriate for binding, and detecting and/or quantifying binding of the peptide to said test substance.

High-throughput screening technologies based on the biomarker, uses and methods of the invention, e.g. configured in an array format, are suitable to monitor biomarker signatures for the identification of potentially useful therapeutic compounds, e.g. ligands such as natural compounds, synthetic chemical compounds (e.g. from combinatorial libraries), peptides, monoclonal or polyclonal antibodies or fragments thereof, which may be capable of binding the biomarker.

Methods of the invention can be performed in array format, e.g. on a chip, or as a multiwell array. Methods can be adapted into platforms for single tests, or multiple identical or multiple non-identical tests, and can be performed in high throughput format. Methods of the invention may comprise performing one or more additional, different tests to confirm or exclude diagnosis, and/or to further characterise a condition.

The invention further provides a substance, e.g. a ligand, identified or identifiable by an identification or screening method or use of the invention. Such substances may be capable of inhibiting, directly or indirectly, the activity of the peptide biomarker, or of suppressing generation of the peptide biomarker. The term “substances” includes substances that do not directly bind the peptide biomarker and directly modulate a function, but instead indirectly modulate a function of the peptide biomarker. Ligands are also included in the term substances; ligands of the invention (e.g. a natural or synthetic chemical compound, peptide, aptamer, oligonucleotide, antibody or antibody fragment) are capable of binding, suitably specific binding, to the peptide.

The invention further provides a substance according to the invention for use in the treatment of schizophrenia or other psychotic disorder, or predisposition thereto.

Also provided is the use of a substance according to the invention in the treatment of schizophrenia or other psychotic disorder, or predisposition thereto.

Also provided is the use of a substance according to the invention as a medicament.

A kit for diagnosing or monitoring schizophrenia or other psychotic disorder, or predisposition thereto is provided. Suitably a kit according to the invention may contain one or more components selected from the group: a ligand specific for the peptide biomarker or a structural/shape mimic of the peptide biomarker, one or more controls, one or more reagents and one or more consumables; optionally together with instructions for use of the kit in accordance with any of the methods defined herein.

The identification of biomarkers for schizophrenia or other psychotic disorder permits integration of diagnostic procedures and therapeutic regimes. Currently there are significant delays in determining effective treatment and hitherto it has not been possible to perform rapid assessment of drug response. Traditionally, many anti-psychotic therapies have required treatment trials lasting weeks to months for a given therapeutic approach. Detection of a peptide biomarker of the invention can be used to screen subjects prior to their participation in clinical trials. The biomarkers provide the means to indicate therapeutic response, failure to respond, unfavourable side-effect profile, degree of medication compliance and achievement of adequate serum drug levels. The biomarkers may be used to provide warning of adverse drug response. Biomarkers are useful in development of personalized brain therapies, as assessment of response can be used to fine-tune dosage, minimise the number of prescribed medications, reduce the delay in attaining effective therapy and avoid adverse drug reactions. Thus by monitoring a biomarker of the invention, patient care can be tailored precisely to match the needs determined by the disorder and the pharmacogenomic profile of the patient, the biomarker can thus be used to titrate the optimal dose, predict a positive therapeutic response and identify those patients at high risk of severe side effects.

Biomarker-based tests provide a first line assessment of ‘new’ patients, and provide objective measures for accurate and rapid diagnosis, in a time frame and with precision, not achievable using the current subjective measures.

Furthermore, diagnostic biomarker tests are useful to identify family members or patients at high risk of developing schizophrenia or other psychotic disorder. This permits initiation of appropriate therapy, or preventive measures, e.g. managing risk factors. These approaches are recognised to improve outcome and may prevent overt onset of the disorder.

Biomarker monitoring methods, biosensors and kits are also vital as patient monitoring tools, to enable the physician to determine whether relapse is due to worsening of the disorder, poor patient compliance or substance abuse. If pharmacological treatment is assessed to be inadequate, then therapy can be reinstated or increased; a change in therapy can be given if appropriate. As the biomarkers are sensitive to the state of the disorder, they provide an indication of the impact of drug therapy or of substance abuse.

The following studies illustrate the invention.

### (a) Study 1

22 serum samples taken from first onset drug-naive schizophrenia patients and 33 controls. These were analyzed using 1 dimensional LC-MS^{E}. The results were then analysed using data analysis software known as Elucidator, which includes sophisticated features for data mining. One of the features includes investigation of post-translational modifications of proteins and peptides which may yield further candidate markers. Data mining and extraction of all possible information out of these discovery studies may be used to enhance the current diagnostic panel for schizophrenia.

Emphasis was made to identify differences in phosphorylation of proteins or peptides, between the patient and control populations. An effort was also made to identify differential cleavage of proteins. Raw data was processed and searched using Proteinlynx Global Server (PLGS). The database was the human Swissprot version 57.4. Raw data was also imported into the Elucidator database. It was then aligned and annotated by importing search results produced by PLGS. Aligned data was then analyzed and a Student's T-Test was used to determine significantly changing proteins.

### (i) Proteins

The significantly changing proteins identified in Study 1 are listed in Table 1:

TABLE1

| | Total | | |
|---|---|---|---|
| | number of | T-Test | |
| | peptides | (P | Ratio |
| Protein Description | detected | value) | patient/control |
| Ig mu chain C region (IgM) | 13 | 0.0026 | 0.69 |
| Leucine zipper-EF-hand- | 3 | 0.0035 | 0.73 |
| containing transmembrane | | | |
| protein 1 | | | |
| Condensin complex subunit | 1 | 0.0043 | 1.62 |
| 2 | | | |
| DNA-directed RNA | 2 | 0.0049 | 1.11 |
| polymerase III subunit RPC5 | | | |
| CD5 antigen-like (SP-alpha) | 4 | 0.0053 | 0.68 |
| Alpha-1-antitrypsin-related | 2 | 0.0226 | 0.89 |
| protein | | | |
| Daple | 2 | 0.026 | 0.75 |
| Apolipoprotein A-I | 36 | 0.0278 | 0.90 |
| Coagulation factor XIII B | 3 | 0.0283 | 0.89 |
| chain | | | |
| Serotransferrin | 28 | 0.0352 | 0.83 |
| B box and SPRY domain- | 2 | 0.0386 | 0.87 |
| containing protein. | | | |
| Apolipoprotein C1 | 2 | 0.0447 | 0.87 |
| Alpha-2-HS-glycoprotein | 14 | 0.0448 | 0.92 |
| (fetuin A) | | | |

### (ii) Peptides

In this section, significant peptides are listed (separately for each protein). These are peptides of proteins that were not statistically significant on the protein level in section (i) and have a sequence that is unique to the protein.

Three peptides belonging to Complement factor H were found statistically significant (out of 81) and these are listed in Table 2:

TABLE 2

| | | T-test- |
|---|---|---|
| Protein Description | Peptide Name | P-value |
| Complement factor H | DHNSNIR | 0.0030 |
| precursor | (SEQ ID NO: 1) | |
| Complement factor H | CVNPPTVQNAYIVSR | 0.0041 |
| precursor | (SEQ ID NO: 2) | |
| Complement factor H | DVYKAGEQVTYTCATYYK | 0.0248 |
| precursor | (SEQ ID NO: 3) | |

Hemopexin was found borderline significant on the protein level with a p value of 0.0740, ratio of 1.05 (p/c). On the peptide level, three were found significantly increased as well (Table 3). There are no known cleavage sites on this protein and no alternative splicing.

TABLE 3

| Protein Description | Peptide Name | T-test-P-value |
|---|---|---|
| Hemopexin precursor | GEFVWK (SEQ ID NO: 4) | 0.0027 |
| (Beta-1B-glycoprotein). | | |
| Hemopexin precursor | ELISER (SEQ ID NO: 5) | 0.0086 |
| (Beta-1B-glycoprotein). | | |
| Hemopexin precursor | LHIMAGR (SEQ ID NO: 6) | 0.0115 |
| (Beta-1B-glycoprotein). | | |

Leucine zipper-EF-hand-containing transmembrane protein 1 (LETM1) is not different on the protein level between patients and controls. However one (of three) peptides (SEQ ID NO: 7) was found to be decreased in the patient population (Table 4). There was no alternative splicing that could explain this finding.

TABLE 4

| | | T-test- | Fold |
|---|---|---|---|
| Protein Description | Peptide Name | P-value | Change |
| Leucine zipper-EF- | LIAEEGVDSLNVK | 0.0053 | 0.63 |
| hand-containing | (SEQ ID NO: 7) | | |
| transmembrane | | | |
| protein 1 | | | |
| Leucine zipper-EF- | GSATKDFSVFFQK | 0.6230 | 0.94 |
| hand-containing | (SEQ ID NO: 8) | | |
| transmembrane | | | |
| protein 1 | | | |
| Leucine zipper-EF- | SEVAKDFEPER | 0.8423 | 0.97 |
| hand-containing | (SEQ ID NO: 9) | | |
| transmembrane | | | |
| protein 1 | | | |

DNA-directed RNA polymerase III subunit (RPC5) was identified based on two peptides. One was found not to be changing and the other is increased (SEQ ID NO: 10) in the patient population (Table 5). There are no alternative splicing that could explain this finding.

TABLE 5

| Protein | | T-test- | Fold |
|---|---|---|---|
| Description | Peptide Name | P-value | Change |
| DNA-directed RNA | VQSYEFLQK | 0.0033 | 1.22 |
| polymerase III | (SEQ ID NO: 10) | | |
| subunit RPC5 | | | |
| DNA-directed RNA | AFVEATFQR | 0.3327 | 1.03 |
| polymerase III | (SEQ ID NO: 11) | | |
| subunit RPC5 | | | |

Synaptotagmin 2 (SYT2) was identified with two peptides (Table 6), one of which is increased (ratio of 1.22, p=0.0073; SEQ ID NO: 12) and one is decreased (ratio of 0.89, p=0.2241; SEQ ID NO: 13). There is no alternative splicing for this protein.

TABLE 6

| Protein | | T-test- | Fold |
|---|---|---|---|
| Description | Peptide Name | P-value | Change |
| Synaptotagmin-2 | MDVGGLSDPYVK | 0.0073 | 1.13 |
| | (SEQ ID NO: 12) | | |
| Synaptotagmin-2 | LTVC[160.0307]ILEAK | 0.2241 | 0.89 |
| | (SEQ ID NO: 13) | | |

(iii) Phosphopeptides

Among the peptides identified in this study were a total of 290 unique phosphopeptides. 12 were found to be statistically significant changing between the patients and controls. They are listed in Table 7. Among these phosphopeptides, only Alpha-2-HS-glycoprotein was found changing on the protein level, and to a lesser extent Complement factor H.

TABLE 7

| | | Known | | Fold |
|---|---|---|---|---|
| | Modified Peptide | phos. | | Change |
| Protein Description | Sequence | site? | P-value | (P/c) |
| Alpha-2-HS- | HTFMGVVSLGS[166.9984] | Yes | 0.003 | 0.835 |
| glycoprotein (Fetuin- | PSGEVSHPR (SEQ ID NO: | | | |
| A) | 14) | | | |
| Complement factor H | LSYT[181.014]C[160.0307] | No | 0.0034 | 1.15 |
| (H factor 1) | EGGFR (SEQ ID NO: 15) | | | |
| Apolipoprotein E | LVQY[243.0296]RGEVQAM | No | 0.0064 | 0.66 |
| | LGQS[166.9984]TEELR | | | |
| | (SEQ ID NO: 16) | | | |
| Inter-alpha-trypsin | YSLFC[160.0307]LGFGFDV | No | 0.0075 | 1.15 |
| inhibitor heavy chain | SY[243.0296]AFLEK (SEQ | | | |
| H4 | ID NO: 17) | | | |
| Ig gamma-2 chain C | EEMTKNQVSLT[181.014]C | No | 0.0084 | 1.32 |
| region. | [160.0307]LVK (SEQ ID | | | |
| | NO: 18) | | | |
| Inter-alpha-trypsin | HPVHRDFLGFY[243.0296] | No | 0.0219 | 1.24 |
| inhibitor heavy chain | VVDSHR (SEQ ID NO: 19) | | | |
| H3 | | | | |
| Zinc-alpha-2- | WEAEPVY[243.0296]VQR | No | 0.0237 | 0.80 |
| glycoprotein | (SEQ ID NO: 20) | | | |
| Apolipoprotein C-III | ASEAEDASLLS[166.9984]F | No | 0.0379 | 0.85 |
| | MQGYMK (SEQ ID NO: 21) | | | |
| Myosin-XVIIIa | VASGS[166.9984]DLHLTD | Yes | 0.043 | 0.84 |
| | IDSDSNR (SEQ ID NO: 22) | | | |
| Inter-alpha-trypsin | T[181.014]LRVQGNDHS | No | 0.0433 | 0.82 |
| inhibitor heavy chain | [166.9984]ATR (SEQ ID | | | |
| H4 | NO: 23) | | | |
| Uncharacterized | Y[243.0296]LIDHPVS | No | 0.0455 | 0.87 |
| protein C1orf125 | [166.9984]LT[181.014] | | | |
| | GAGR (SEQ ID NO: 24) | | | |
| Complement C4-A | VTASDPLDTLGS[166.9984] | No | 0.0461 | 1.12 |
| | EGALS[166.9984]PGGVAS | | | |
| | [166.9984]LLR (SEQ ID | | | |
| | NO: 25) | | | |

### (b) Study 2

Study 2 was conducted in an analogous manner to that described in Study 1, except that 20 serum samples taken from first onset drug-naive schizophrenia patients and 17 controls. These were analyzed using 2 dimensional LC-MS^{E}.

### (i) Proteins

The significantly changing proteins identified in Study 2 are listed in Table 8:

TABLE 8

| Protein Description | p value | Peptide Count | Fold Change |
|---|---|---|---|
| Lumican | 0.0043 | 20 | −1.12 |
| Synemin | 0.0062 | 15 | 1.11 |
| Collagen alpha-1(V) chain | 0.0082 | 3 | 1.21 |
| Leucine-rich repeat-containing | 0.0084 | 7 | −1.15 |
| protein 16A | | | |
| Microtubule-associated | 0.0128 | 4 | 1.26 |
| protein 2 | | | |
| Apolipoprotein L1 | 0.0133 | 7 | 1.16 |
| Haptoglobin | 0.0159 | 44 | 1.41 |
| Vang-like protein 1 | 0.0179 | 2 | −1.30 |
| Gelsolin | 0.0204 | 54 | −1.07 |
| Alpha-actinin-2 | 0.0225 | 4 | −1.21 |
| Probable ATP-dependent DNA | 0.0226 | 2 | −1.29 |
| helicase HFM1 | | | |
| Fibulin-1 | 0.0237 | 4 | −1.24 |
| DNA-directed RNA polymerase | 0.0245 | 4 | −1.24 |
| III subunit RPC1 | | | |
| CDK5 regulatory subunit- | 0.0264 | 2 | 1.17 |
| associated protein 2 | | | |
| RANBP2-like and GRIP | 0.0264 | 7 | 1.32 |
| domain-containing protein 1 | | | |
| Putative zinc-alpha-2- | 0.0266 | 2 | −1.24 |
| glycoprotein-like 1 | | | |
| Coagulation factor XIII B | 0.0374 | 25 | −1.12 |
| chain | | | |
| Retinol-binding protein 4 | 0.0393 | 18 | −1.08 |
| Alpha-2-antiplasmin | 0.0438 | 20 | 1.09 |
| Dedicator of cytokinesis | 0.0445 | 12 | −1.18 |
| protein 3 | | | |
| Myoferlin | 0.0480 | 5 | −1.23 |
| Peroxisome proliferator- | 0.0016 | 1 | 1.54 |
| activated receptor gamma | | | |
| coactivator-related protein 1 | | | |
| Phosphorylated CTD- | 0.0038 | 1 | 1.80 |
| interacting factor 1 | | | |
| Inactive ubiquitin carboxyl- | 0.0090 | 1 | −1.66 |
| terminal hydrolase 54 | | | |
| Serine/threonine-protein | 0.0094 | 1 | −1.46 |
| kinase 38-like | | | |
| Ig kappa chain V-I region | 0.0119 | 1 | −1.30 |
| WEA | | | |
| Neurexin-1-alpha | 0.0131 | 1 | 1.16 |
| Prostate-specific membrane | 0.0143 | 1 | −1.46 |
| antigen-like protein | | | |
| Mitochondrial dicarboxylate | 0.0154 | 1 | 1.51 |
| carrier | | | |
| Ig kappa chain V-III region | 0.0157 | 1 | −2.04 |
| SIE | | | |
| Creatine kinase | 0.0157 | 1 | −1.62 |
| Protein GREB1 | 0.0262 | 1 | −1.49 |
| Seprase | 0.0367 | 1 | 1.31 |
| Pre-mRNA-processing factor 6 | 0.0372 | 1 | −1.30 |
| Spermatogenesis-associated | 0.0439 | 1 | 1.22 |
| protein 21 | | | |
| Protein SOLO | 0.0476 | 1 | −1.56 |
| Apolipoprotein F | 0.0490 | 1 | −1.77 |

### (ii) Peptides

In this section, significant peptides (p<0.01) are listed (separately for each protein). These are peptides of proteins that were not statistically significant on the protein level in section (i) and have a sequence that is unique to the protein.

One peptide belonging to Apoliprotein M was identified as differentially expressed out of a total of 4 peptides of this 21 kDa protein. The significant peptide is listed in Table 9:

TABLE 9

| | | p | Fold |
|---|---|---|---|
| Protein Description | Peptide Name | value | Change |
| Apolipoprotein M | AFLLTPRNQEACELSNN | 0.0065 | −1.50 |
| (Apo-M) ApoM) | (SEQ ID NO: 26) | | |
| ((Protein G3a) | | | |

One peptide belonging to Kininogen 1 was found statistically significant (out of 74) and is listed in Table 10:

TABLE 10

| Protein | | p | Fold |
|---|---|---|---|
| Description | Peptide Name | value | Change |
| Kininogen-1 | SVSEINPTTQMK | 0.0024 | 1.82 |
| | (SEQ ID NO: 27) | | |

Four Inter-alpha-trypsin inhibitor heavy chain H4 (ITIH4) peptides were identified as differentially expressed (out of a total of 102 identified) and are listed in Table 11:

TABLE 11

| Protein Description | Peptide Name | p value | Fold Change |
|---|---|---|---|
| Inter-alpha-trypsin inhibitor | NPLVWVH | 0.0038 | 1.44 |
| heavy chain H4 | (SEQ ID NO: 28) | | |
| Inter-alpha-trypsin inhibitor | ANTVQEATFQMELPKK | 0.0047 | −1.40 |
| heavy chain H4 | (SEQ ID NO: 29) | | |
| Inter-alpha-trypsin inhibitor | NGIDIYSLTVDSR | 0.0089 | 1.14 |
| heavy chain H4 | (SEQ ID NO: 30) | | |
| Inter-alpha-trypsin inhibitor | LALDNGGLAR | 0.0099 | 1.26 |
| heavy chain H4 | (SEQ ID NO: 31) | | |

Three of the significant peptides are upregulated and one is down regulated. There is no data indicating this is a result of alternative splicing nor do these span a cleavage site.

Two Complement component C8 gamma chain (CO8G) peptides were identified as differentially expressed, a third one was borderline significant showing similar expression difference as the other two. A total of 15 peptides were identified. The significant peptides are listed in Table 12:

TABLE 12

| | | | Fold |
|---|---|---|---|
| Protein Description | Peptide Name | p value | Change |
| Complement component C8 | AEATTLHVAPQGTAMAVSTFR | 0.0039 | −1.59 |
| gamma chain | (SEQ ID NO: 32) | | |
| Complement component C8 | LDGICWQVRQLYGDTGVLGR | 0.0133 | −1.49 |
| gamma chain | (SEQ ID NO: 33) | | |
| Complement component C8 | KLDGICWQVR | 0.0927 | −1.51 |
| gamma chain | (SEQ ID NO: 34) | | |

One NACHT and WD repeat domain-containing protein 1 (NWD1) peptide was identified as differentially expressed out of a total of 18 peptides. The significant peptide is listed in Table 13:

TABLE 13

| | | | Fold |
|---|---|---|---|
| Protein Description | Peptide Name | p value | Change |
| NACHT and WD repeat | GITAMAWGVEEK | 0.00415 | 1.43 |
| domain-containing | (SEQ ID NO: 35) | | |
| protein 1. | | | |

Two Annexin A6 (ANXA6) peptides were identified as differentially expressed of 3 peptides identified. The significant peptides are listed in Table 14:

TABLE 14

| Protein | | | Fold |
|---|---|---|---|
| Description | Peptide Name | p value | Change |
| Annexin A6 | YELTGKFER | 0.00491 | −1.65 |
| | (SEQ ID NO: 36) | | |
| Annexin A6 | EDAQVAAEILEIADTPSGDK | 0.0350 | −1.45 |
| | (SEQ ID NO: 37) | | |

Two PROS peptides were identified as differentially expressed and four other borderline significant of 32 peptides identified. The significant peptides are listed in Table 15:

TABLE 15

| Protein | | | Fold |
|---|---|---|---|
| Description | Peptide Name | p value | Change |
| Vitamin | ASFTCTCKPGWQGEKCEFDI | 0.0091 | −1.34 |
| K-dependent | NECK (SEQ ID NO: 38) | | |
| protein S | | | |
| precursor | | | |
| Vitamin | SCVNAIPDQCSPLPCNEDGY | 0.0438 | −1.28 |
| K-dependent | MSCK (SEQ ID NO: 39) | | |
| protein S | | | |
| precursor | | | |
| Vitamin | FSAEFDFR | 0.0547 | −1.39 |
| K-dependent | (SEQ ID NO: 40) | | |
| protein S | | | |
| precursor | | | |
| Vitamin | EEAREVFENDPETDYFYPK | 0.0684 | −1.25 |
| K-dependent | (SEQ ID NO: 41) | | |
| protein S | | | |
| precursor | | | |
| Vitamin | HNDIRAHSCPSVWK | 0.0742 | −1.37 |
| K-dependent | (SEQ ID NO: 42) | | |
| protein S | | | |
| precursor | | | |
| Vitamin | KVESELIKPINPR | 0.0758 | −1.36 |
| K-dependent | (SEQ ID NO: 43) | | |
| protein S | | | |
| precursor | | | |

(iii) Phosphopeptides

Among the peptides identified in this study were a total of 1649 unique phosphopeptides. 11 were found to be statistically significant (p<0.01) changing between the patients and controls and are listed in Table 16.

TABLE 16

| | | | Fold |
|---|---|---|---|
| Protein Description | Peptide Name | p value | Change |
| Vitamin K-dependent | LAQDQKSCEVVS[p]VCLPLNLDT[p]K | 0.000959 | −1.9427 |
| protein S | (SEQ ID NO: 44) | | |
| Inter-alpha-trypsin | GFIDGHYKDY[p]FVPQLYSFLK | 0.001925 | −1.37693 |
| inhibitor heavy chain | (SEQ ID NO: 45) | | |
| H2 | | | |
| C4b-binding protein | TDLSFGS[p]QIEFSCSEGFFLIGSTTSR | 0.002797 | −1.60653 |
| alpha chain | (SEQ ID NO: 46) | | |
| Ig heavy chain V-III | GLEWVSAIGT[p]AGDQYYADSVK | 0.004396 | −1.55815 |
| region BRO | (SEQ ID NO: 47) | | |
| Complement factor | LQNNENNISCVERGWS[p]TPPK | 0.005194 | 2.15044 |
| H-related protein 2 | (SEQ ID NO: 48) | | |
| Haptoglobin | DIAPT[p]LTLY[p]VGKK | 0.005487 | −1.63253 |
| | (SEQ ID NO: 49) | | |
| ProthrombinThrombin | IS[p]MLEKIY[p]IHPR | 0.006444 | 1.756426 |
| heavy chain | (SEQ ID NO: 50) | | |
| Complement | SYTSHT[p]NEIHK | 0.006761 | 1.570047 |
| component C7 | (SEQ ID NO: 51) | | |
| Beta-2-glycoprotein 1 | ATVVY[p]QGERVK (SEQ ID NO: 52) | 0.006805 | −1.98581 |
| Complement factor H | IPCSQPPQIEHGT[p]INSS[p]R | 0.008304 | −1.46466 |
| | (SEQ ID NO: 53) | | |
| Histidine-rich | HPNVFGFCRADLFY[p]DVEALDLES[p]PK | 0.008946 | −1.36983 |
| glycoprotein | (SEQ ID NO: 54) | | |

### (c) Study 3

Study 3 was conducted in an analogous manner to that described in Study 1 at the protein level. The significantly changing proteins identified in Study 3 are listed in Table 17:

TABLE 17

| | Total | | |
|---|---|---|---|
| | number of | | |
| | peptides | T-Test | Ratio |
| Protein Description | detected | (p value) | patient/control |
| CD5L | 3 | 0.0014 | 0.44 (2.28 down) |
| IgM | 5 | 0.0016 | 0.48 (2.08 down) |
| Coagulation Factor | 4 | 0.014 | 0.82 (1.22 down) |
| XIII B | | | |
| Transferrin | 31 | 0.0199 | 0.73 (1.37 down) |
| Apolipoprotein D | 6 | 0.0241 | 0.83 (1.2 down) |
| Apolipoprotein A1 | 29 | 0.0302 | 0.79 (1.27 down) |
| Alpha-2-HS- | 21 | 0.0366 | 0.86 (1.16 down) |
| glycoprotein | | | |
| Apolipoprotein A4 | 29 | 0.0412 | 0.82 (1.22 down) |
| Apolipoprotein A2 | 9 | 0.0422 | 0.81 (1.23 down) |
| Apolipoprotein Cl | 2 | 0.0444 | 0.81 (1.23 down) |

1. Use of Complement factor H or a functional fragment or phosphorylated fragment thereof, as a biomarker for schizophrenia or other psychotic disorder, or predisposition thereto.

2. Use as defined in claim 1, wherein the functional fragment is selected from any of the following peptides:
(SEQ ID NO: 1)
DHNSNIR;
(SEQ ID NO: 2)
CVNPPTVQNAYIVSR;
and
(SEQ ID NO: 3)
DVYKAGEQVTYTCATYYK.

3. Use as defined in claim 1, wherein the phosphorylated fragment is selected from any of the following phosphopeptides:
(SEQ ID NO: 15)
LSYT[181.014]C[160.0307]EGGFR
and
(SEQ ID NO: 53)
IPCSQPPQIEHGT[p]INSS[p]R.

4. Use as defined in any of claims 1 to 3, which additionally comprises one or more analytes selected from: Condensin complex subunit 2, DNA-directed RNA polymerase III subunit RPC5, Daple, Coagulation factor XIII B chain, B box and SPRY domain-containing protein, Synaptotagmin-2, Zinc-alpha-2-glycoprotein, Uncharacterised protein C1orf125, Synemin, Collagen alpha-1(V) chain, Leucine-rich repeat-containing protein 16A, Vang-like protein 1, Alpha-actinin-2, Probable ATP-dependent DNA helicase HFM1, Fibulin-1, DNA-directed RNA polymerase III subunit RPC1, RANBP2-like and GRIP domain-containing protein 1, Putative zinc-alpha-2-glycoprotein-like 1, Alpha-2-antiplasmin, Dedicator of cytokinesis protein 3, Phosphorylated CTD-interacting factor 1, Inactive ubiquitin carboxyl-terminal hydrolase 54, Serine/threonine protein kinase 38-like, Ig kappa chain V-I region WEA, Ig kappa chain V-III region SIE, Protein GREB1, Seprase, Pre-mRNA-processing factor 6, Spermatogenesis-associated protein 21 (SPAT21), Protein SOLO, Apolipoprotein F, Apolipoprotein M, Kininogen-1 (KNG1), Complement component 8 gamma chain (CO8G), NACHT and WD repeat domain-containing protein 1 (NWD1), Annexin A6 (ANXA6), Ig heavy chain V-III region BRO, Complement factor H-related protein 2, Prothrombin Thrombin heavy chain, Histidine-rich glycoprotein, Ig gamma-2 chain C region, Mitochondrial dicarboxylate protein, CD5 ligand, Transferrin, Apolipoprotein D, Apolipoprotein A2, Apolipoprotein A4, Ig mu chain C region (IgM), Leucine zipper-EF-hand-containing transmembrane protein 1, CD5 antigen-like (SP-alpha), Alpha-1-antitrypsin-related protein, Apolipoprotein A1, Serotransferrin, Apolipoprotein C1, Alpha-2-HS-glycoprotein (fetuin A), Hemopexin precursor (Beta-1B-glycoprotein), Apolipoprotein C-III, Apolipoprotein E, Inter-alpha-trypsin inhibitor heavy chain H4, Inter-alpha-trypsin inhibitor heavy chain H3, Myosin-XVIIIa, Complement C4-A, Lumican, Microtubule-associated protein 2, Apolipoprotein L1, Haptoglobin, Gelsolin, CDK5 regulatory subunit-associated protein 2, Retinol-binding protein 4 (RBP4), Myoferlin, Peroxisome proliferator-activated receptor gamma coactivator-related protein 1, Neurexin-1-alpha, Prostate-specific membrane antigen-like protein, Creatine kinase, Vitamin K-dependent protein S precursor, Inter-alpha-trypsin inhibitor heavy chain H2, C4b-binding protein alpha chain, Complement component C7 and Beta-2-glycoprotein 1 (Apolipoprotein H), or a functional fragment or phosphorylated derivative thereof.

5. Use as defined in claim 4, wherein the analyte is selected from Condensin complex subunit 2, DNA-directed RNA polymerase III subunit RPC5, Daple, Coagulation factor XIII B chain, B box and SPRY domain-containing protein, Synemin, Collagen alpha-1(V) chain, Leucine-rich repeat-containing protein 16A, Vang-like protein 1, Alpha-actinin-2, Probable ATP-dependent DNA helicase HFM1, Fibulin-1, DNA-directed RNA polymerase III subunit RPC1, RANBP2-like and GRIP domain-containing protein 1, Putative zinc-alpha-2-glycoprotein-like 1, Alpha-2-antiplasmin, Dedicator of cytokinesis protein 3, Phosphorylated CTD-interacting factor 1, Inactive ubiquitin carboxyl-terminal hydrolase 54, Serine/threonine protein kinase 38-like, Ig kappa chain V-I region WEA, Mitochondrial dicarboxylate protein, Ig kappa chain V-III region SIE, Protein GREB1, Seprase, Pre-mRNA-processing factor 6, Spermatogenesis-associated protein 21 (SPAT21), Protein SOLO and Apolipoprotein F.

6. Use as defined in claim 4, wherein the analyte is selected from: DNA-directed RNA polymerase III subunit RPC5, Complement factor H, Synaptotagmin-2, Apolipoprotein M, Kininogen-1 (KNG1), Complement component 8 gamma chain (CO8G), NACHT and WD repeat domain-containing protein 1 (NWD1) and Annexin A6 (ANXA6) or a functional fragment thereof.

7. Use as defined in claim 6, wherein the functional fragment thereof comprises one or more peptides selected from SEQ ID NOS: 1-3, 10-13, 26-27, and 32-37.

8. Use as defined in claim 4, wherein the analyte is selected from: Complement factor H, Ig gamma-2 chain C region, Zinc-alpha-2-glycoprotein, Uncharacterised protein C1orf125, Ig heavy chain V-III region BRO, Complement factor H-related protein 2, Prothrombin Thrombin heavy chain and Histidine-rich glycoprotein or a phosphorylated derivative thereof.

9. Use as defined in claim 8, wherein the phosphorylated derivative thereof comprises one or more peptides selected from SEQ ID NOS: 15, 18, 20, 24, 47-48, 50, 53-54, which contains 1, 2 or 3, such as 1 or 2 phosphorylated amino acids.

10. Use as defined in claim 4, which comprises any of the peptides selected from SEQ ID NOs 1 to 54, or a phosphorylated derivative thereof.

11. Use of Complement factor H, Condensin complex subunit 2, DNA-directed RNA polymerase III subunit RPC5, Daple, Coagulation factor XIII B chain, B box and SPRY domain-containing protein, Synaptotagmin-2, Zinc-alpha-2-glycoprotein, Uncharacterised protein C1orf125, Synemin, Collagen alpha-1(V) chain, Leucine-rich repeat-containing protein 16A, Vang-like protein 1, Alpha-actinin-2, Probable ATP-dependent DNA helicase HFM1, Fibulin-1, DNA-directed RNA polymerase III subunit RPC1, RANBP2-like and GRIP domain-containing protein 1, Putative zinc-alpha-2-glycoprotein-like 1, Alpha-2-antiplasmin, Dedicator of cytokinesis protein 3, Phosphorylated CTD-interacting factor 1, Inactive ubiquitin carboxyl-terminal hydrolase 54, Serine/threonine protein kinase 38-like, Ig kappa chain V-I region WEA, Ig kappa chain V-III region SIE, Protein GREB1, Seprase, Pre-mRNA-processing factor 6, Spermatogenesis-associated protein 21 (SPAT21), Protein SOLO, Apolipoprotein F, Apolipoprotein M, Kininogen-1 (KNG1), Complement component 8 gamma chain (CO8G), NACHT and WD repeat domain-containing protein 1 (NWD1), Annexin A6 (ANXA6), Ig heavy chain V-III region BRO, Complement factor H-related protein 2, Prothrombin Thrombin heavy chain, Histidine-rich glycoprotein, Ig gamma-2 chain C region, Mitochondrial dicarboxylate protein, CD5 ligand, Transferrin, Apolipoprotein D, Apolipoprotein A2, Apolipoprotein A4, Ig mu chain C region (IgM), Leucine zipper-EF-hand-containing transmembrane protein 1, CD5 antigen-like (SP-alpha), Alpha-1-antitrypsin-related protein, Apolipoprotein A1, Serotransferrin, Apolipoprotein C1, Alpha-2-HS-glycoprotein (fetuin A), Hemopexin precursor (Beta-1B-glycoprotein), Apolipoprotein C-III, Apolipoprotein E, Inter-alpha-trypsin inhibitor heavy chain H4, Inter-alpha-trypsin inhibitor heavy chain H3, Myosin-XVIIIa, Complement C4-A, Lumican, Microtubule-associated protein 2, Apolipoprotein L1, Haptoglobin, Gelsolin, CDK5 is regulatory subunit-associated protein 2, Retinol-binding protein 4 (RBP4), Myoferlin, Peroxisome proliferator-activated receptor gamma coactivator-related protein 1, Neurexin-1-alpha, Prostate-specific membrane antigen-like protein, Creatine kinase, Vitamin K-dependent protein S precursor, Inter-alpha-trypsin inhibitor heavy chain H2, C4b-binding protein alpha chain, Complement component C7 and Beta-2-glycoprotein 1 (Apolipoprotein H), as a specific panel of analyte biomarkers for schizophrenia or other psychotic disorder, or predisposition thereto.

12. A method of diagnosing schizophrenia or other psychotic disorder, or predisposition in an individual thereto, comprising:
(a) obtaining a biological sample from an individual;
(b) quantifying the amounts of the analyte biomarkers as defined in any of claims 1 to 11;
(c) comparing the amounts of the analyte biomarkers in the biological sample with the amounts present in a normal control biological sample from a normal subject, such that a difference in the level of the analyte biomarkers in the biological sample is indicative of schizophrenia or other psychotic disorder, or predisposition thereto.

13. A method of monitoring efficacy of a therapy in a subject having, suspected of having, or of being predisposed to schizophrenia or other psychotic disorder, comprising detecting and/or quantifying, in a sample from said subject, the analyte biomarkers as defined in any of claims 1 to 11.

14. A method as defined in claim 12 or claim 13, which is conducted on samples taken on two or more occasions from a test subject.

15. A method as defined in any of claims 12 to 14, further comprising comparing the level of the biomarker present in samples taken on two or more occasions.

16. A method as defined in any of claims 12 to 15, comprising comparing the amount of the biomarker in said test sample with the amount present in one or more samples taken from said subject prior to commencement of therapy, and/or one or more samples taken from said subject at an earlier stage of therapy.

17. A method as defined in any of claims 12 to 16, further comprising detecting a change in the amount of the biomarker in samples taken on two or more occasions.

18. A method as defined in any of claims 12 to 17, comprising comparing the amount of the biomarker present in said test sample with one or more controls.

19. A method as defined in claim 18, comprising comparing the amount of the biomarker in a test sample with the amount of the biomarker present in a sample from a normal subject.

20. A method as defined in any of claims 12 to 19, wherein samples are taken prior to and/or during and/or following therapy for schizophrenia or other psychotic disorder.

21. A method as defined in any of claims 12 to 20, wherein samples are taken at intervals over the remaining life, or a part thereof, of a subject.

22. A method as defined in any of claims 12 to 21, wherein quantifying is performed by measuring the concentration of the analyte biomarker in the or each sample.

23. A method as defined in any of claims 12 to 22, wherein detecting and/or quantifying is performed by one or more methods selected from SELDI (-TOF), MALDI (-TOF), a 1-D gel-based analysis, a 2-D gel-based analysis, Mass spec (MS), reverse phase (RP) LC, size permeation (gel filtration), ion exchange, affinity, HPLC, UPLC or other LC or LC-MS-based technique.

24. A method as defined in any of claims 12 to 23, wherein detecting and/or quantifying is performed using an immunological method.

25. A method as defined in any of claims 12 to 24, wherein the detecting and/or quantifying is performed using a biosensor or a microanalytical, microengineered, microseparation or immunochromatography system.

26. A method as defined in any of claims 12 to 25, wherein the biological sample is cerebrospinal fluid, whole blood, blood serum, plasma, urine, saliva, or other bodily fluid, or breath, condensed breath, or an extract or purification therefrom, or dilution thereof.

27. A kit for monitoring or diagnosing schizophrenia or other psychotic disorder, comprising a biosensor capable of detecting and/or quantifying the analyte biomarkers as defined in any of claims 1 to 11.
